(19) United States
(12) Patent Application Publication
(10) Pub. No.: US 2010/0316733 A1
(43) Pub. Date: Dec. 16, 2010

(54) Hyperbaric Oxygen Therapy and Treatment Method

(76) Inventor: Kenneth R. Locklear, West Palm Beach, FL (US)

Correspondence Address: BROOKS KUSHMAN P.C., 1000 TOWN CENTER, TWENTY-SECOND FLOOR, SOUTHFIELD, MI, 48075, US

(21) Appl. No.: 12/815,735

(22) Filed: Jun. 15, 2010

Related U.S. Application Data
(60) Provisional application No. 61/187,072, filed on Jun. 15, 2009.

(51) Int. Cl. A61K 33/30; A61G 10/02; A61K 33/00; A61K 33/06; A61K 33/04; A61P 35/00

(52) U.S. Cl. 424/641; 128/205.26; 424/600; 424/682; 424/702

(57) ABSTRACT

A method of treating cancer concurrent with receiving chemotherapy treatments which may effectively ameliorate the anemic and toxic effects induced by chemotherapy includes prescribing at least three hyperbaric oxygen therapy treatments per week at a pressure of at least 1.5 ATA, the treatments lasting at least 30 minutes each, prescribing a diet including less than about 300 grams of carbohydrates a day, and prescribing nutritional supplements in accordance with a nutritional supplement regimen including elevated amounts of vitamin B-12, vitamin E, and zinc as compared to a reference daily intake.

### CROSS-REFERENCE TO RELATED APPLICATIONS

This application claims the benefit of U.S. provisional Application No. 61/187,072 filed Jun. 15, 2009, the disclosure of which is incorporated in its entirety by reference herein.

### TECHNICAL FIELD

This invention relates to a hyperbaric oxygen therapy and treatment method for treating cancer concurrent with receiving chemotherapy.

### BACKGROUND

Cancer is a common and serious disease in which abnormal cells divide uncontrollably, often forming tumors. There are over 100 types of cancer, affecting various organ and cell types within the body. One of the most common treatments for cancer is chemotherapy.

Chemotherapy is a treatment that uses drugs to kill cancer cells, thereby eliminating the cancer or slowing down the tumor growth. Currently, most chemotherapy drugs are not able to target only cancer cells, so some normal, healthy cells are also destroyed during treatment. This leads to side effects, such as fatigue, nausea, vomiting, decreased red blood cell counts (anemia), decreased white blood cell counts (neutropenia), hair loss, mouth sores, and pain. Chemotherapy is given in a number of different ways, including intravenously, by injection, intra-arterially, orally (pill form), and topically. The dose and length of the treatment is determined based on the type of cancer and the drug prescribed, and may be adjusted based on the patient reaction. Typically, chemotherapy is administered in cycles, wherein each cycle includes a treatment period and a rest period. Cycles can vary in length, and multiple cycles may be given based on the drug, type of cancer, and patient response.

While chemotherapy is effective in many cases for cancer treatment, a protocol which reduces the side effects and toxicity of the treatment would be beneficial.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

The present invention includes a method of treating cancer comprising receiving chemotherapy, undergoing hyperbaric oxygen therapy (HBOT), consuming a low carbohydrate diet, and taking a combination of nutritional supplements. This method may effectively reduce the anemic and toxic effects induced by chemotherapy, thus allowing for increased dosing or frequency of chemotherapy treatments.

HBOT is a treatment in which a patient is enclosed in a pressurized chamber containing pure oxygen at elevated pressure for a period of time sufficient to cause the desired increase in oxygen concentration in the body. The increased oxygen concentration and pressure allows more oxygen to be absorbed into the bloodstream. HBOT can be done multiple times per week, often at times up to about 2 hours. The air inside the chamber is typically 100% oxygen at a pressure up to 3.0 ATA (atmospheres absolute). Typically, HBOT is used for ailments such as decompression sickness and carbon monoxide poisoning, but recently research has been done to see if HBOT can assist in the cancer treatment process.

Research has shown that cancerous tumors create a hypoxic environment (low oxygen concentration), which can hinder the effectiveness of drugs used in chemotherapy. Without being bound to any particular theory, it is believed that HBOT is able to increase the oxygen concentration in the tumor environment, allowing the chemotherapy to be more effective. Also, it is believed that HBOT can help to maintain a more normal red and white blood cell count during chemotherapy, which normally causes a reduction in both cell types. This stabilization can reduce or eliminate the need for erythropoietin-stimulating agents, which are given to increase red blood cell counts. These benefits, as well as a reduction in other side effects, contribute to a reduction in toxicity and can help allow a more aggressive chemotherapy treatment than might normally be tolerated by a patient.

According to an aspect of the present invention, HBOT may be prescribed concurrently with chemotherapy in the treatment of cancer. The HBOT can be performed at a pressure from about 1.0 ATA to 3.0 ATA, in another embodiment about 1.5 ATA to 2.75 ATA, and in another embodiment about 2.0 ATA to 2.5 ATA. The time per treatment may vary from about 0 to 120 minutes, in another embodiment from about 30 to 110 minutes, and in another embodiment from about 45 to 100 minutes. The number of treatments per week may vary from 1 to 7, in another embodiment from 2 to 6, and in another embodiment from 4 to 5. The pressure, time per treatment, and number of treatments per week may vary from patient to patient and treatment to treatment depending on the patient's tolerance level of the HBOT, type of cancer, type of drug, stage of treatment, or other factors considered by the patient's doctor, and the values and examples provided herein are not intended to be limiting. Days of treatment need not be consecutive, and the individual treatment settings can be changed from day to day and week to week and need not be constant throughout the course of treatment.

Most tumors receive their energy from the body in the form of glucose, a sugar/carbohydrate, and not from fat. Without being bound to any particular theory, it is believed that a diet low in carbohydrates will effectively “starve” a tumor by not providing it enough energy to continue its rapid growth. The patient may replace this energy with more fats and proteins, therefore allowing the rest of the healthy cells in the body to function normally.

In accordance with one aspect of the present invention, a low carbohydrate diet and low sugar protocol may be prescribed in addition to chemotherapy. The protocol comprises at least one time period concurrent with a time period during which chemotherapy is being received in which carbohydrate consumption is limited to an amount substantially below the daily recommended value, which is currently 300 grams. According to one aspect of the present invention, there may be at least two time periods concurrent with chemotherapy with lowered carbohydrate and sugar consumption, wherein the upper limit of carbohydrates consumed is different in consecutive time periods. According to another aspect of the present invention, there may be at least three time periods concurrent with chemotherapy with lowered carbohydrate and sugar consumption, wherein the upper limit of carbohydrates consumed is different in consecutive time periods. If there are multiple time periods, then the upper limit of carbohydrates consumed during each period may progressively increase from the first period to the last. After the completion of chemotherapy, the patient may continue to consume a low carbohydrate diet, especially if the tumor was not completely eliminated.

According to one aspect of the present invention, there are three time periods concurrent with the time period during which chemotherapy is being received. The first time period may begin substantially at a time that the chemotherapy treatments start and lasts for about 20 to 40 days, the second time period may begin at the end of the first time period and lasts for about 20 to 40 days, and the third time period may begin at the end of the second time period and last for the remainder of the chemotherapy treatments. According to an aspect of the present invention, the periods may be the first 30 days of chemotherapy, days 31-60 of chemotherapy, and days 61 until the end of chemotherapy. Carbohydrate consumption may be limited to less than about 20 grams of carbohydrates per day during the first period, less than about 40 grams of carbohydrates per day during the second period, and less than about 100 grams of carbohydrates per day during the third period.

Of course, it is understood that the present invention is not limited to these carbohydrate values and time periods. The length of the time periods and the upper limit of carbohydrates consumed per day in each time period may vary depending on the total length of chemotherapy treatment and the number of time periods chosen. According to one aspect of the present invention, carbohydrate consumption may be limited to no more than about 30 grams of carbohydrates per day during the first period and carbohydrate consumption per day may be limited so as to not exceed about 150 grams during a period that is concurrent with chemotherapy.

Prescribing a regimen of nutritional supplements is also contemplated in accordance with an aspect of the present invention, to be taken concurrently with chemotherapy. Without being held to any particular theory, it is believed that nutritional supplements can help to improve the body's response to chemotherapy, help alleviate some of the symptoms, fight tumor progression, and rebuild cells damaged by chemotherapy. The regimen may comprise one or more of calcium, vitamin B-12, vitamin D, vitamin E, zinc, flax seed oil, and selenium. The amounts taken of each supplement will generally surpass the Reference Daily Intake (RDI) for that supplement, as provided by the FDA.

According to one aspect of the present invention, daily calcium and Vitamin D intake may be between about 1,000 and 2,400 mg, and in one embodiment about 2,400 mg. Daily vitamin B-12 intake may be between about 500 and 1,500 mg, in one embodiment about 750 and 1,250 mg, and in one embodiment about 1,000 mg. Daily vitamin E intake may be between about 50 and 1,000 IU, in one embodiment between about 250 and 900 IU, and in one embodiment about 800 IU. Daily zinc intake may be between about 15 and 100 mg, in one embodiment between about 25 and 75 mg, and in one embodiment about 50 mg. Daily flax seed oil intake may be between about 250 and 2,000 mg, in one embodiment between about 500 and 1,500 mg, and more in one embodiment about 1,000 mg. Daily selenium intake may be between about 100 and 1,000 μg, in one embodiment between about 250 and 750 μg, and in one embodiment about 400 μg.

It is understood that the nutritional supplement regimen and values described above are not meant to be limiting. The supplements contemplated above need not all be taken, individual supplements may be removed or others not explicitly contemplated herein may be added. Furthermore, the intake of each supplement may deviate from day to day within the ranges given. Supplements may be taken all at once or as several smaller doses throughout the day. For example, vitamin E could be taken as 400 IU, twice per day or selenium could be taken as 200 μg, twice per day. For some supplements, taking smaller doses multiple times per day may be advantageous.

While the HBOT, diet protocol, and nutritional supplement regimen can be separately combined with chemotherapy in any manner (e.g., concurrently, sequentially, etc.), it is contemplated according to the present invention that all three are used concurrently with the chemotherapy. If one or more of the HBOT, diet protocol, and nutritional supplement regimen produce unexpected and/or undesirable side effects, then the problematic component(s) of the treatment may be removed, either temporarily or permanently.

In summary, according to one aspect of the present invention, a method of treating cancer concurrent with receiving chemotherapy treatments may be provided. The method may include undergoing at least three hyperbaric oxygen therapy treatments per week at a pressure of at least 1.5 ATA, the treatments lasting at least 30 minutes each, consuming a diet including less than 300 grams of carbohydrates a day in at least one time period concurrent with receiving chemotherapy, and taking nutritional supplements in accordance with a nutritional supplement regimen, the regimen including taking elevated amounts of vitamin B-12, vitamin E, and zinc as compared to the reference daily intake. The nutritional supplement regimen may further comprise taking at least one of calcium and vitamin D, flax seed oil, and selenium.

In accordance with one aspect of the present invention, the hyperbaric oxygen therapy may be received five times per week at a pressure of at least about 2.0 ATA and for at least 60 minutes per treatment. Successive time periods concurrent with receiving chemotherapy may include a diet with a progressively higher upper limit of carbohydrates consumed per day than a previous time period. For example, there may be three time periods, the diet of the first time period including an upper limit of about 20 grams of carbohydrates per day, the diet of the second time period including an upper limit of about 40 grams of carbohydrates per day, and the diet of the third time period including an upper limit of about 100 grams of carbohydrates per day. The first time period may begin substantially at a time that the chemotherapy treatments start and last for about 30 days, the second time period may begin at the end of the first time period and last for about 30 days, and the third time period may start at the end of the second time period and last for the remainder of the chemotherapy treatments.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method of treating cancer concurrent with receiving chemotherapy treatments, the method comprising:
prescribing at least three hyperbaric oxygen therapy treatments per week at a pressure of at least 1.5 ATA, the treatments lasting at least 30 minutes each;
prescribing a diet including less than about 300 grams of carbohydrates a day; and
prescribing nutritional supplements in accordance with a nutritional supplement regimen including elevated amounts of vitamin B-12, vitamin E, and zinc as compared to a reference daily intake.

2. The method of claim 1, wherein there are at least two time periods concurrent with the chemotherapy treatments, and the diet in a first time period has a different upper limit of carbohydrates consumed per day compared to the diet in a second time period.

3. The method of claim 2, wherein the diet for each time period has a progressively higher upper limit of carbohydrates consumed per day than the diet for a previous time period.

4. The method of claim 3, wherein there are three time periods, the diet of the first time period including an upper limit of about 20 grams of carbohydrates per day, the diet of the second time period including an upper limit of about 40 grams of carbohydrates per day, and the diet of a third time period including an upper limit of about 100 grams of carbohydrates per day.

5. The method of claim 4, wherein the first time period begins substantially at a time that the chemotherapy treatments start and lasts for about 20 to 40 days, the second time period begins at the end of the first time period and lasts for about 20 to 40 days, and the third time period starts at the end of the second time period and lasts for the remainder of the chemotherapy treatments.

6. The method of claim 5, wherein the first and second time periods each last for about 30 days.

7. The method of claim 1, wherein the hyperbaric oxygen therapy is prescribed five times per week at a pressure of at least about 2.0 ATA and for at least about 60 minutes per treatment.

8. The method of claim 1, wherein the nutritional supplement regimen further comprises at least one of calcium, flax seed oil, and selenium.

9. The method of claim 8, wherein the nutritional supplement regimen comprises about 1,000 to 2,400 mg of calcium per day, about 1,000 to 2,400 mg of vitamin D per day, about 500 to 1,500 mg of vitamin B-12 per day, about 50 to 1000 IU of vitamin E per day, about 15 to 100 mg of zinc per day, about 250 to 2,000 mg of flax seed oil per day, and about 100 to 1,000 μg of selenium per day.

10. A method of treating cancer concurrent with receiving chemotherapy treatments, the method comprising:
prescribing at least three hyperbaric oxygen therapy treatments per week at a pressure of at least 1.5 ATA, the treatments lasting at least 30 minutes each;
prescribing a diet having a different upper limit of carbohydrates per day in each of at least two time periods concurrent with the chemotherapy, the diet for each time period including less than about 300 grams of carbohydrates a day; and
prescribing nutritional supplements in accordance with a nutritional supplement regimen including elevated amounts of at least one of calcium, vitamin B-12, vitamin D, vitamin E, zinc, flax seed oil, and selenium as compared to a reference daily intake.

11. The method of claim 10, wherein the diet for each time period has a progressively higher upper limit of carbohydrates consumed per day than the diet for a previous time period.

12. The method of claim 10, wherein the at least two time periods includes a first time period that begins substantially at a time that the chemotherapy treatments start and lasts for about 20 to 40 days and a second time period that begins at the end of the first time period and lasts for about 20 to 40 days.

13. The method of claim 12, wherein the first and second time periods each last for about 30 days.

14. The method of claim 12, wherein the diet of the first time period includes an upper limit of about 20 grams of carbohydrates per day and the diet of the second time period includes an upper limit of about 40 grams of carbohydrates per day.

15. The method of claim 12, wherein the at least two time periods further includes a third time period starting from the end of the second time period and lasting for the remainder of the chemotherapy treatments.

16. The method of claim 15, wherein a diet of the third time period includes an upper limit of about 100 grams of carbohydrates per day.

17. The method of claim 10, wherein the hyperbaric oxygen therapy is prescribed five times per week at a pressure of at least about 2.0 ATA and for at least about 60 minutes per treatment.

18. The method of claim 10, wherein the nutritional supplement regimen comprises about 500 to 1,500 mg of vitamin B-12 per day, about 50 to 1000 IU of vitamin E per day, and about 15 to 100 mg of zinc per day.

19. The method of claim 18, wherein the nutritional supplement regimen further comprises about 1,000 to 2,400 mg of calcium per day, about 1,000 to 2,400 mg of vitamin D per day, about 250 to 2,000 mg of flax seed oil per day, and about 100 to 1,000 μg of selenium per day.

20. A method of treating cancer concurrent with receiving chemotherapy treatments, the method comprising:
prescribing at least three hyperbaric oxygen therapy treatments per week at a pressure of at least about 1.5 ATA, the treatments lasting at least about 30 minutes each;
prescribing a diet in a first time period with an upper limit of carbohydrates of about 20 grams per day starting with the beginning of chemotherapy and lasting for about 20 to 40 days, a diet in a second time period with an upper limit of carbohydrates of about 40 grams per day starting with the end of the first time period and lasting for about 20 to 40 days, and a diet in a third time period with an upper limit of carbohydrates of about 100 grams per day starting with the end of the second time period and lasting for the remainder of the chemotherapy; and
prescribing nutritional supplements in accordance with a nutritional supplement regimen including about 1,000 to 2,400 mg of calcium per day, about 750 to 1,250 mg of vitamin B-12 per day, about 1,000 to 2,400 mg of vitamin D per day, about 250 to 900 IU of vitamin E per day, about 25 to 75 mg of zinc per day, about 500 to 1,500 mg of flax seed oil per day, and about 250 to 750 μg of selenium per day.
